(19) United States
(12) Patent Application Publication
(10) Pub. No.: US 2023/0230677 A1
(43) Pub. Date: Jul. 20, 2023

(54) TREATMENT SUPPORT APPARATUS, TREATMENT SUPPORT METHOD, AND TREATMENT SUPPORT PROGRAM

(71) Applicant: FUJIFILM Corporation, Tokyo (JP)

(72) Inventor: Azuma TAKAHASHI, Tokyo (JP)

(73) Assignee: FUJIFILM Corporation, Tokyo (JP)

(21) Appl. No.: 18/176,488

(22) Filed: Mar. 1, 2023

Related U.S. Application Data
(63) Continuation of application No. PCT/JP2021/030803, filed on Aug. 23, 2021.

(30) Foreign Application Priority Data
Sep. 28, 2020 (JP) 2020-162673

(51) Int. Cl. G16H 30/20; G16H 50/50; A61B 5/00; G06T 7/00

(52) U.S. Cl. CPC G16H 30/20; G16H 50/50; A61B 5/4848; G06T 7/0016; G06T 2207/30101; G06T 2207/20081

(57) ABSTRACT

A processor searches for a first similar case from among a plurality of reference cases, each of the plurality of reference cases including at least one diagnosed image and a diagnosis log, the diagnosis log describing a treatment method performed on a diagnosed patient for whom the diagnosed image is acquired and a treatment result obtained by the treatment method, the first similar case having a similar feature to a target image obtained by imaging a treatment target patient who is to be treated, the first similar case including, as the diagnosed image, a post-treatment image obtained through imaging after treatment. The processor further searches for a second similar case from among the plurality of reference cases, the second similar case having a similar feature to the post-treatment image included in the first similar case. Further, the processor presents the treatment method and the treatment result described in a search diagnosis log that is the diagnosis log included in each of the first similar case and the second similar case.

### CROSS-REFERENCE TO RELATED APPLICATIONS

The present application is a Continuation of PCT International Application No. PCT/JP2021/030803, filed on Aug. 23, 2021, which claims priority to Japanese Patent Application No. 2020-162673, filed on Sep. 28, 2020. Each application above is hereby expressly incorporated by reference, in its entirety, into the present application.

### BACKGROUND

### Technical field

The present disclosure relates to a treatment support apparatus, a treatment support method, and a treatment support program.

### Related Art

A similar case search apparatus that searches for a similar case image similar to an image of a treatment target from among a plurality of case images has been proposed. Techniques have also been proposed for referring to a diagnosis log included in a found similar case to acquire information such as a treatment method performed on the similar case and a result of the treatment (see JP2015-203920A and JP2013-146327A).

The techniques described in JP2015-203980A and JP2013-146327A provide a result of treatment by the treatment method that has been performed, that is, a treatment result. However, merely referring to treatment result of similar cases makes it difficult to provide a doctor with information that fully reflects the effects of various treatment methods on the treatment results.

### SUMMARY OF THE INVENTION

The present disclosure has been made in consideration of the circumstances described above, and aims to provide information that fully reflects the effect of a treatment method on a treatment result.

A treatment support apparatus according to the present disclosure includes at least one processor.

The processor is configured to search for a first similar case from among a plurality of reference cases, each of the plurality of reference cases including at least one diagnosed image and a diagnosis log, the diagnosis log describing a treatment method performed on a diagnosed patient for whom the diagnosed image is acquired and a treatment result obtained by the treatment method, the first similar case having a similar feature to a target image obtained by imaging a treatment target patient who is to be treated, the first similar case including, as the diagnosed image, a post-treatment image obtained through imaging after treatment; search for a second similar case from among the plurality of reference cases, the second similar case having a similar feature to the post-treatment image included in the first similar case; and present the treatment method and the treatment result described in a search diagnosis log that is the diagnosis log included in each of the first similar case and the second similar case.

The term “diagnosed image” means an image acquired by imaging a patient who has been diagnosed and treated for a disease until the treatment is completed. Accordingly, the diagnosed image includes a pre-treatment image acquired by imaging the patient before the treatment and a post-treatment image acquired by imaging the patient after the treatment. The term “diagnosed patient” refers to a patient for whom a diagnosed image is acquired and for whom treatment is completed.

The term “treatment method” is used to include, for example, surgery, medication, and the like.

In the treatment support apparatus according to the present disclosure, the diagnosis log may describe doctor-responsible-for-diagnosis information and patient information, the doctor-responsible-for-diagnosis information identifying a doctor who has given treatment to the diagnosed patient, the patient information including a diagnosis result of the diagnosed patient before the treatment, and the processor may be configured to further present the doctor-responsible-for-diagnosis information and the patient information.

In the treatment support apparatus according to the present disclosure, the processor may be configured to derive an expected treatment result on the basis of treating-doctor information identifying a doctor in charge of treatment for the treatment target patient, patient information including a diagnosis result of the treatment target patient, and the treatment method described in the search diagnosis log, the expected treatment result being a treatment result expected if the doctor in charge of treatment treats the treatment target patient in accordance with the treatment method described in the search diagnosis log.

In the treatment support apparatus according to the present disclosure, the processor may be configured to derive the expected treatment result by using a trained model, the trained model being constructed by machine learning using training data in which treating-doctor information, patient information, and treatment methods are training information and treatment results are ground-truth data.

In the treatment support apparatus according to the present disclosure, in a case where a plurality of portions of treatment are present in the treatment target patient and a new target image is acquired by imaging the treatment target patient each time treatment is performed on one of the portions of treatment, the processor may be configured to repeat a new search for the first similar case, a new search for the second similar case, and presentation of the treatment result each time treatment is performed on one of the portions of treatment.

In the treatment support apparatus according to the present disclosure, the target image may be acquired by imaging a treatment target patient having stenosis in a coronary artery, the treatment method may include a type of a stent, a placement position of the stent, and a dilation diameter of the coronary artery, and the treatment result may be a diameter of the coronary artery after placement of the stent.

A treatment support method according to the present disclosure searches for a first similar case from among a plurality of reference cases, each of the plurality of reference cases including at least one diagnosed image and a diagnosis log, the diagnosis log describing a treatment method performed on a diagnosed patient for whom the diagnosed image is acquired and a treatment result obtained by the treatment method, the first similar case having a similar feature to a target image obtained by imaging a treatment target patient who is to be treated, the first similar case including, as the diagnosed image, a post-treatment image obtained through imaging after treatment; searches for a second similar case from among the plurality of reference cases, the second similar case having a similar feature to the post-treatment image included in the first similar case; and presents the treatment method and the treatment result described in a search diagnosis log that is the diagnosis log included in each of the first similar case and the second similar case.

A program for causing a computer to execute the treatment support method according to the present disclosure may be provided.

According to the present disclosure, information that fully reflects the effect of a treatment method on a treatment result can be provided.

### BRIEF DESCRIPTION OF THE DRAWINGS

FIG. 1 is a diagram illustrating a schematic configuration of a medical information system to which a treatment support apparatus according to an embodiment of the present disclosure is applied;

FIG. 2 is a diagram schematically illustrating a file configuration of reference cases;

FIG. 3 is a diagram illustrating a schematic configuration of the treatment support apparatus according to the embodiment;

FIG. 4 is a functional configuration diagram of the treatment support apparatus according to the embodiment;

FIG. 5 is a diagram schematically illustrating a search for a first similar case and a second similar case;

FIG. 6 is a diagram illustrating the contents of the description of a diagnosis log;

FIG. 7 is a diagram illustrating an example of a pre-treatment image and a post-treatment image;

FIG. 8 is a diagram illustrating training data;

FIG. 9 is a diagram illustrating a presentation screen;

FIG. 10 is a diagram illustrating the contents of a table included in the presentation screen;

FIG. 11 is a flowchart illustrating a process performed in the embodiment; and

FIG. 12 is a flowchart illustrating a process performed in another embodiment.

### DETAILED DESCRIPTION

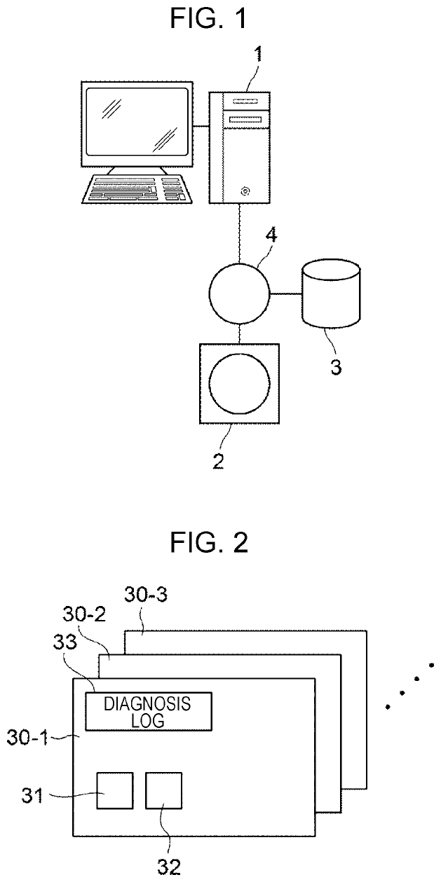

The following describes an embodiment of the present disclosure with reference to the drawings. First, a configuration of a medical information system to which a treatment support apparatus according to this embodiment is applied will be described. FIG. 1 is a diagram illustrating a schematic configuration of the medical information system. In the medical information system illustrated in FIG. 1, a computer 1 including the treatment support apparatus according to this embodiment, an imaging device 2, and an image storage server 3 are connected so as to be capable of communicating with each other via a network 4.

The computer 1 includes the treatment support apparatus according to this embodiment, and has installed therein a treatment support program according to this embodiment. The computer 1 may be a workstation or a personal computer to be directly operated by a doctor who performs diagnosis, or may be a server computer connected to the workstation or personal computer via a network. The treatment support program is stored in a storage device of a server computer connected to a network or a network storage in such a manner that the treatment support program is externally accessible. The treatment support program is downloaded to and installed in the computer 1 used by the doctor in response to a request. Alternatively, the treatment support program is recorded and distributed on a recording medium such as a DVD (Digital Versatile Disc) or a CD-ROM (Compact Disc Read Only Memory), and is installed in the computer 1 from the recording medium.

The imaging device 2 is a device that captures an image of a treatment-target site of a subject to generate an image indicating the site. Specifically, the imaging device 2 is a device that acquires a three-dimensional image, such as a CT (Computed Tomography) device, an Mill (Magnetic Resonance Imaging) device, or a PET (Positron Emission Tomography) device. Alternatively, the imaging device 2 may be an ultrasound imaging device that acquires an ultrasound image or a radiographic imaging device that acquires a radiographic image of the subject. The image generated by the imaging device 2 is transmitted to and stored in the image storage server 3.

The image storage server 3 is a computer that stores and manages various kinds of data. The image storage server 3 includes a large-capacity external storage device and database management software. The image storage server 3 communicates with the computer 1 and other devices via the network 4, which is wired or wireless, and transmits and receives image data and so on. Specifically, various kinds of data including image data of the image generated by the imaging device 2 are acquired via a network, and are stored and managed in a recording medium such as a large-capacity external storage device. The storage format of the image data and the communication between the devices via the network 4 are based on a protocol such as DICOM (Digital Imaging and Communication in Medicine).

In this embodiment, the image storage server 3 stores a plurality of reference cases. FIG. 2 is a diagram schematically illustrating a file configuration of the reference cases stored in the image storage server 3. As illustrated in FIG. 2, the image storage server 3 stores a plurality of reference cases 30-1, 30-2, 30-3, etc. In the following description, one reference case to be referred to is represented by reference numeral 30. The reference case 30 includes a diagnosed image acquired in the process of treatment for one patient who has completed treatment (i.e., a diagnosed patient). The diagnosed image includes a pre-treatment image 31 acquired by imaging before the treatment and a post-treatment image 32 acquired by imaging after the treatment. The reference case 30 may include only one of the pre-treatment image 31 and the post-treatment image 32. The reference case 30 may include a plurality of pre-treatment images 31. The reference case 30 may include a plurality of post-treatment images 32. The pre-treatment image 31 and the post-treatment image 32 may be acquired by different types of imaging devices 2.

The post-treatment image 32 includes an image acquired immediately after treatment (for example, immediately after surgery), and also includes a follow-up image acquired after a certain period of time has elapsed after treatment to check the degree of treatment. One reference case 30 is associated with a diagnosis log 33 acquired in the process of treatment. In FIG. 2, the diagnosis log 33 is included in the file of the reference case 30. Alternatively, the diagnosis log 33 and the reference case 30 may be stored in the image storage server 3 as separate files associated with each other.

In this embodiment, the pre-treatment image 31 and the post-treatment image 32 included in one reference case 30 each constitute one image file. In this case, the tag of the image file describes information on the imaging date and time and the name of the patient. For example, when the image is a three-dimensional image, the tag of the image file also describes information on the number of slices and the slice interval. The number of slices and the slice interval are also described in the diagnosis log 33.

In this embodiment, the image storage server 3 also stores a target image G0 obtained by imaging a treatment target patient to be treated. In this embodiment, a diagnosis log is also generated for the target image G0 and is stored in the image storage server 3 in association with the target image G0. Image diagnosis for the target image G0 has completed, and diagnosis results are described in the diagnosis log. By contrast, no treatment has been given to the treatment target patient for whom the target image G0 is acquired.

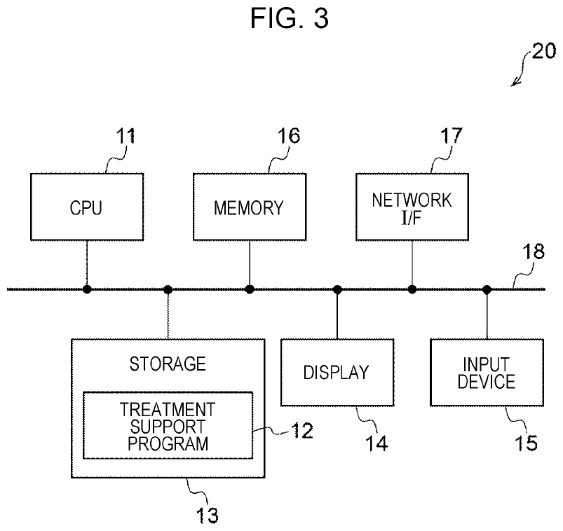

Next, the treatment support apparatus according to this embodiment will be described. FIG. 3 illustrates a hardware configuration of the treatment support apparatus according to this embodiment. As illustrated in FIG. 3, a treatment support apparatus 20 includes a CPU (Central Processing Unit) 11, a nonvolatile storage 13, and a memory 16 serving as a temporary storage area. The treatment support apparatus 20 further includes a display 14 such as a liquid crystal display, an input device 15 such as a keyboard and a mouse, and a network I/F (Interface) 17 to be connected to the network 4. The CPU 11, the storage 13, the display 14, the input device 15, the memory 16, and the network I/F 17 are connected to a bus 18. The CPU 11 is an example of a processor in the present disclosure.

The storage 13 is implemented by an HDD (Hard Disk Drive), an SSD (Solid State Drive), a flash memory, and so on. The storage 13 serving as a storage medium stores a treatment support program 12. The CPU 11 reads the treatment support program 12 from the storage 13, loads the treatment support program 12 onto the memory 16, and executes the loaded treatment support program 12.

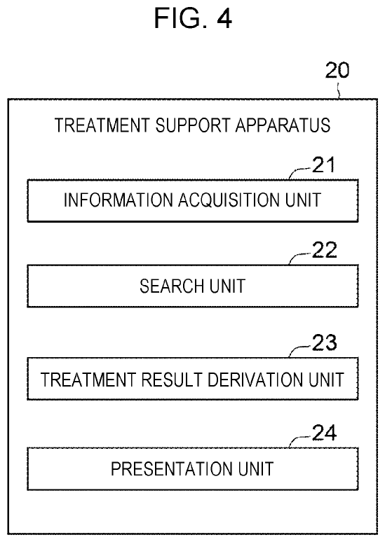

Next, a functional configuration of the treatment support apparatus according to this embodiment will be described. FIG. 4 is a diagram illustrating a functional configuration of the treatment support apparatus according to this embodiment. As illustrated in FIG. 4, the treatment support apparatus 20 includes an information acquisition unit 21, a search unit 22, a treatment result derivation unit 23, and a presentation unit 24. The CPU 11 executes the treatment support program 12. As a result, the CPU 11 functions as the information acquisition unit 21, the search unit 22, the treatment result derivation unit 23, and the presentation unit 24.

The information acquisition unit 21 acquires the target image G0, which is obtained by imaging the treatment target patient, from the image storage server 3 via the network I/F 17 in accordance with an instruction given by an operator such as a doctor through the input device 15. In this embodiment, a CT image obtained by imaging the breast of a patient with stenosis at a coronary artery bifurcation using a CT apparatus is acquired as the target image G0. Accordingly, the target image G0 is constituted by a plurality of tomographic images. In a case where the target image G0 has already been stored in the storage 13, the information acquisition unit 21 may acquire the target image G0 from the storage 13. In this embodiment, as described above, image diagnosis with interpretation of the target image G0 has been completed.

The search unit 22 searches for a similar case having a similar feature to the target image G0 acquired by the information acquisition unit 21 from among the plurality of reference cases stored in the image storage server 3. For this purpose, the search unit 22 identifies features of the target image G0. The features of the target image G0 include the number of slices included in the target image G0, the slice interval, an imaging site in the target image G0, and the target image G0 itself.

The target image G0 is stored in the storage 13 as one image file. The image file is assigned tag information. The tag information describes the imaging date and time of the target image G0, the imaging site, the number of slices, the slice interval, and so on. The search unit 22 refers to the tag information assigned to the image file of the target image G0 and acquires the imaging site, the number of slices, and the slice interval of the target image G0. The search unit 22 may refer to the diagnosis log 33 and acquire the imaging site, the number of slices, and the slice interval of the target image G0.

In this embodiment, since the target image G0 is acquired by imaging the breast of the patient, information on the imaging site may be acquired by input or the like performed by the operator using the input device 15.

To search for a similar case, the search unit 22 refers to the tag information of the diagnosed images included in the reference cases 30 stored in the image storage server 3 and identifies reference cases 30 including diagnosed images obtained by imaging the same site as a site included in the target image G0. The reference cases 30 identified in this way are referred to as first reference cases.

Then, the search unit 22 identifies, from among the first reference cases, reference cases including diagnosed images having a slice interval that matches that of the target image G0. For example, the slice interval of the target image G0 is 1 mm. In this case, reference cases including diagnosed images having a slice interval of 1 mm are identified. The reference cases identified in this way are referred to as second reference cases. Then, the search unit 22 identifies, from among the second reference cases, reference cases including diagnosed images having a similar number of slices to the target image G0. The reference cases identified in this way are referred to as third reference cases. The phrase “having a similar number of slices” means having slices the number of which falls within a predetermined range (for example, ±10%) relative to the number of slices of the target image G0.

Then, the search unit 22 derives a first similarity S1 based on a correlation value between the target image G0 and each of diagnosed images included in the third reference cases. For example, the target image G0 and each of the diagnosed images are aligned with each other, and the absolute value of the difference between the pixel values of corresponding pixels in the two images after alignment is calculated as the correlation value. Then, the correlation value is normalized to a value ranging from 0 to 1 to derive the first similarity S1. In this embodiment, the target site of treatment is the coronary arteries. Thus, the search unit 22 may perform a region detection process on the target image G0 for search to extract the heart region from the target image G0. The search unit 22 has a trained model such as a neural network trained by machine learning to detect hearts. The search unit 22 uses the trained model to extract the heart from the target image G0. In place of the trained model, template matching using a template indicating the shape of an anatomical feature of the heart may be performed to extract the heart. In this case, the heart may also be extracted from each of the diagnosed images included in the third reference cases, and the first similarity S1 may be derived between the hearts extracted from the two images.

The search unit 22 sorts the diagnosed images in descending order of the first similarity S1. Then, a reference case including a predetermined number of one or more diagnosed images having a large first similarity S1 and including a post-treatment image as a diagnosed image is searched for as a first similar case RS1.

In this embodiment, a CT image of a patient with stenosis at a coronary artery bifurcation is set as the target image G0. For this reason, the first similar case RS1 includes, as a diagnosed image, a CT image of a patient with stenosis at a coronary artery bifurcation, which is similar to the target image G0. In a case where the first similar case RS1 includes the pre-treatment image 31 as a diagnosed image, the pre-treatment image 31 shows the state of stenosis of the coronary arteries before treatment. For treatment of coronary artery stenosis, surgery is performed to place a stent in the stenosis section. For this reason, the post-treatment image 32 included in the first similar case RS1 shows dilatation of the blood vessels due to the placement of the stent in the stenosis section of the coronary arteries.

The pre-treatment image 31 is typically a CT image for accurate diagnosis of the patient. By contrast, the post-treatment image 32 is acquired immediately after surgery and is any image in which the state of the placed stent can be checked. Thus, the post-treatment image 32 is typically a two-dimensional radiographic image acquired by simple radiography. In this embodiment, the pre-treatment image 31 is a CT image like the target image G0, and the post-treatment image 32 is a two-dimensional radiographic image. The two-dimensional radiographic image is referred to simply as a radiographic image.

The search unit 22 further searches for a second similar case RS2 having a similar feature to the post-treatment image 32 included in the first similar case RS1. For this purpose, the search unit 22 refers to the diagnosis log 33 included in the first similar case RS1 and identifies reference cases in a manner similar to that for searching for the first similar case RS1. Specifically, the search unit 22 identifies reference cases including, as diagnosed images, post-treatment images 32 obtained by imaging the same site as that of the post-treatment image 32 included in the first similar case RS1. In a case where the post-treatment image 32 is a CT image, the search unit 22 identifies reference cases that include post-treatment images 32 including the same site and having the same slice interval as the post-treatment image 32 included in the first similar case RS1 and having a similar number of slices to the post-treatment image 32 included in the first similar case RS1. Then, the search unit 22 derives, as a second similarity S2, a correlation value between the post-treatment image included in the first similar case RS1 and each of diagnosed images included in the identified reference cases.

The search unit 22 sorts the diagnosed images in descending order of the second similarity S2. Then, a reference case including a predetermined number of one or more diagnosed images having a large second similarity S2 is searched for as the second similar case RS2.

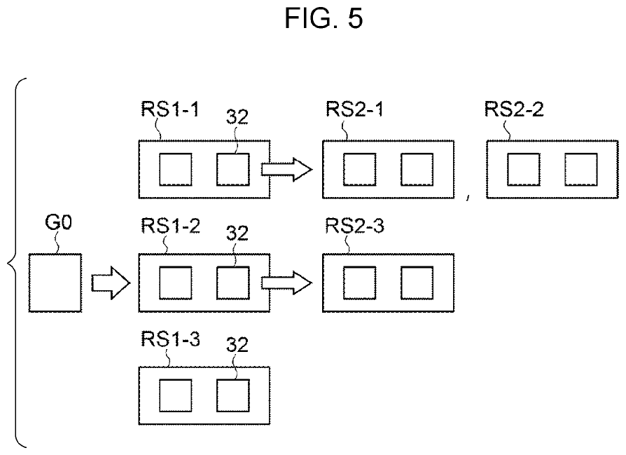

FIG. 5 is a diagram schematically illustrating a search for a first similar case and a second similar case. As illustrated in FIG. 5, in this embodiment, first, the search unit 22 searches for a reference case similar to the target image G0 as a first similar case RS1. In FIG. 5, three first similar cases RS1-1, RS1-2, and RS1-3 have been found.

The search unit 22 further searches for a second similar case RS2 having a similar feature to the post-treatment image 32 included in each of the first similar cases RS1-1, RS1-2, and RS1-3. In FIG. 5, two second similar cases RS2-1 and RS2-2 are found for the post-treatment image 32 in the first similar case RS1-1, one second similar case RS2-3 is found for the post-treatment image 32 in the first similar case RS1-2, and no second similar case RS2 is found for the post-treatment image 32 in the first similar case RS1-3.

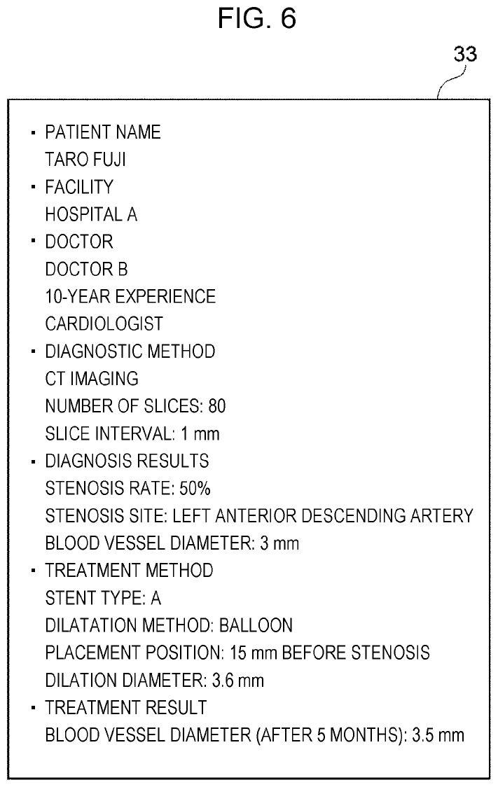

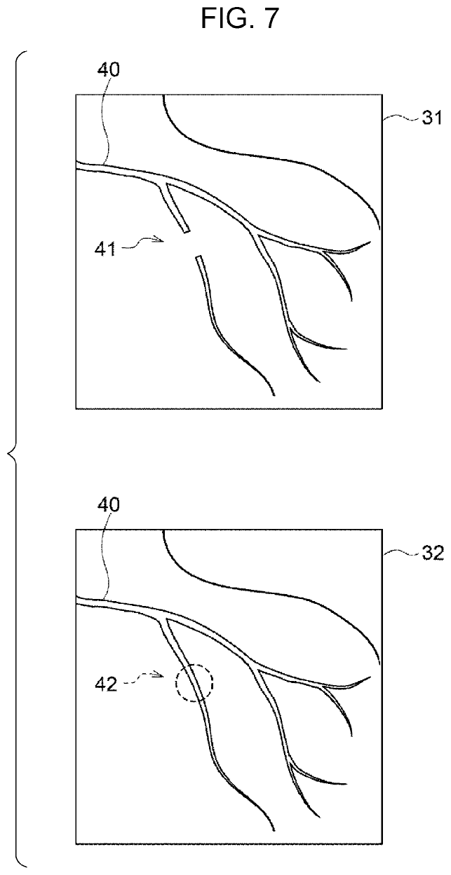

The description of the diagnosis log 33 will now be described. FIG. 6 is a diagram illustrating the contents of the description of a diagnosis log. As illustrated in FIG. 6, the diagnosis log 33 describes, for a reference case, a patient name, a facility where treatment was performed, a doctor who performed treatment, a diagnostic method, diagnosis results, a treatment method, and a treatment result. The doctor who performed treatment corresponds to a doctor responsible for diagnosis.

In the diagnosis log 33 illustrated in FIG. 6, Taro Fuji is described as the patient name. Hospital A is described as the facility. Doctor B and the experience (10 years) and field of expertise (cardiologist) of Doctor B are described as the doctor. Information on the facility and information on the doctor are collectively referred to as environment information. CT imaging is described as the diagnostic method, and the number of slices (80) of a CT image acquired by CT imaging and the slice interval (1 mm) are described. The stenosis rate (50%), the stenosis site (left anterior descending artery), and the blood vessel diameter (3 mm) are described as the diagnosis results. The stent type (A), the dilatation method (balloon), the placement position of the stent (15 mm before stenosis), and the dilation diameter (3.6 mm) due to the stent are described as the treatment method. Further, the blood vessel diameter after 5 months (3.5 mm) is described as the treatment result.

The diagnosis results in the diagnosis log 33 are described on the basis of image diagnosis using the pre-treatment image 31. The treatment result is described on the basis of image diagnosis using the post-treatment image 32. FIG. 7 is a diagram illustrating an example of a pre-treatment image and a post-treatment image. As illustrated in FIG. 7, the pre-treatment image 31 includes coronary arteries 40 of the heart. A stenosis 41 is found in the coronary arteries 40. A doctor who performs diagnosis interprets the pre-treatment image 31, measures a blood vessel diameter, identifies a stenosis site, calculates a stenosis rate, and describes these in a diagnosis log. By contrast, as illustrated in FIG. 7, it is found that, as a result of placement of the stent in the stenosis 41 of the coronary arteries in the pre-treatment image 31, in the post-treatment image 32, the stenosis at a position 42 where the stenosis 41 was present is resolved. The doctor who performs diagnosis compares the pre-treatment image 31 and the post-treatment image 32, measures the blood vessel diameter at the position 42 where the stent has been placed in the post-treatment image 32, and describes the measured blood vessel diameter in the diagnosis log 33 as a treatment result.

Of the diagnosis logs 33 included in the reference cases, diagnosis logs included in the first similar case RS1 and the second similar case RS2 that are found are referred to as a search diagnosis log 33A. In this embodiment, the description of the search diagnosis log 33A is presented, as described below. Specifically, facility information, doctor information, a patient name, diagnosis results (a stenosis rate, a stenosis site, and a blood vessel diameter), and the stent type, the placement position, and the dilation diameter in the treatment method, which are described in the search diagnosis log 33A, are presented. In the following description, the patient name and the diagnosis results (the stenosis rate, the stenosis site, and the blood vessel diameter) are collectively referred to as patient information.

In this embodiment, a diagnosis log is also generated for the target image G0. However, no treatment has been given to the treatment target patient. Accordingly, the diagnosis log for the target image G0 includes only the environment information (i.e., the facility and the doctor) and the patient information (i.e., the patient name and the diagnosis results). The information on the doctor described in the diagnosis log for the target image G0 corresponds to treating-doctor information.

The treatment result derivation unit 23 derives a treatment result expected if the treatment target patient is treated in accordance with the treatment method described in the search diagnosis log 33A. To derive the expected treatment result, the treatment result derivation unit 23 refers to the diagnosis log for the target image G0 and acquires environment information including a facility such as a hospital and a doctor that give treatment to the treatment target patient. Further, the treatment result derivation unit 23 acquires patient information including the patient name of the treatment target patient and the diagnosis results. The treatment result derivation unit 23 further acquires treatment methods described in search diagnosis logs 33A of similar cases.

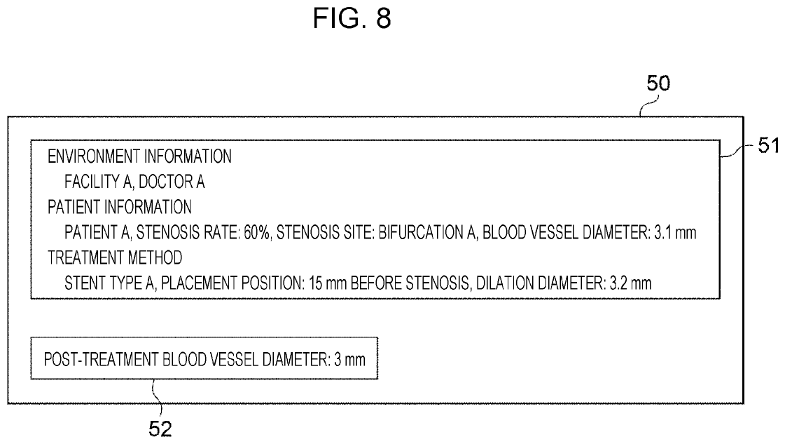

In this embodiment, the treatment result derivation unit 23 derives a treatment result by using a trained model constructed by machine learning using training data. In the training data, the environment information, the patient information, and the treatment method are training information, and the treatment result is ground-truth data. FIG. 8 is a diagram illustrating an example of training data. As illustrated in FIG. 8, training data 50 includes, as training information 51, environment information, patient information, and a treatment method. The environment information includes facility A and doctor A. The patient information includes patient A, a stenosis rate of 60%, bifurcation A as a stenosis site, and a blood vessel diameter of 3.1 mm. The treatment method includes stent (A) as the stent type, 15 mm before stenosis as the placement position of the stent, and a dilation diameter of 3.2 mm. The training data 50 includes, as ground-truth data 52, a post-treatment blood vessel diameter of 3 mm.

A machine learning model can be used as the trained model. Examples of the machine learning model include a neural network model. Examples of the neural network model include a simple perceptron, a multilayer perceptron, a deep neural network, a convolutional neural network, a deep belief network, a recurrent neural network, and a probabilistic neural network.

A neural network for constructing a trained model is trained to output a blood vessel diameter, which is an expected treatment result, when environment information, patient information, and a treatment method are input. Specifically, training information is input to a neural network to output a blood vessel diameter, a difference between the output blood vessel diameter and the blood vessel diameter in the ground-truth data is derived as a loss, and training is repeatedly executed such that the loss approaches 0. As a result, a trained model is constructed. The trained model is installed in the computer 1 together with the treatment support program according to this embodiment.

The presentation unit 24 presents the contents of the description of the search diagnosis log 33A and the expected treatment result to the operator by displaying the contents of the description and the expected treatment result on the display 14. The contents of the description to be presented in the description of the search diagnosis log 33A are the environment information, the patient information, the treatment method, and the treatment result. Environment information and patient information for the patient to be treated are also presented.

To present the contents of the description of the search diagnosis log 33A and the expected treatment result, the presentation unit 24 classifies the first similar case RS1 and the second similar case RS2. To be specific, the first similar case RS1 and the second similar case RS2 are classified by the unit of the post-treatment image 32 included in the first similar case RS2, which is referred to for searching for the second similar case RS1. For example, as illustrated in FIG. 5, the three first similar cases RS1-1, RS1-2, and RS1-3 are found, the two second similar cases RS2-1 and RS2-2 are found using the post-treatment image 32 included in the first similar case RS1-1, the second similar case RS2-3 is found using the post-treatment image 32 included in the first similar case RS1-2, and no second similar case is found for the first similar case RS1-3.

In this case, the presentation unit 24 classifies the first similar cases and the second similar cases RS1 and RS2 into a first group GR1 including the first similar case RS1-1 and the two second similar cases RS2-1 and RS2-2, a second group GR2 including the first similar case RS1-2 and the second similar case RS2-3, and a third group GR3 including only the first similar case RS1-3. Then, the presentation unit 24 presents the contents of the description of the search diagnosis log 33A and the expected treatment result for each of the classified groups.

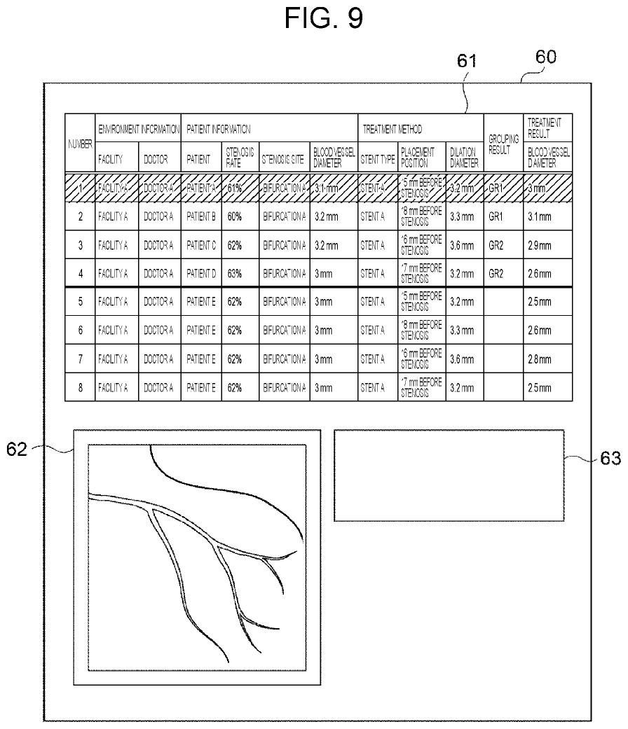

FIG. 9 is a diagram illustrating a presentation screen. In FIG. 9, for convenience of description, search results for similar cases are different from those illustrated in FIG. 5. As illustrated in FIG. 9, a presentation screen 60 displays a table 61 including, for each of patients A to E, environment information, patient information, a treatment method, a grouping result, and a treatment result. An image display region 62 and a text input region 63 are displayed below the table 61.

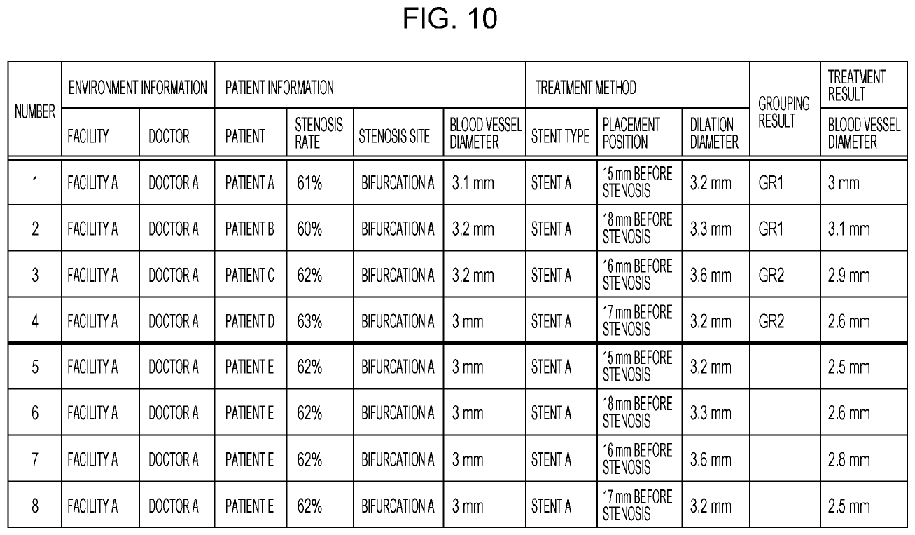

FIG. 10 is a diagram illustrating information described in a table. In FIG. 10, four similar cases including the first similar cases RS1 and the second similar cases RS2 have been found. As illustrated in FIG. 10, rows with numbers 1 to 4 indicate information on similar cases, and rows with numbers 5 to 8 indicate information on a treatment target. The patients A to D are diagnosed patients for whom the found first and second similar cases RS1 and RS2 are acquired, and the patient E is a treatment target patient. The environment information includes a facility and a doctor. The patient information includes a patient name and diagnosis results of the patient, namely, a stenosis rate, a stenosis site, and a blood vessel diameter before treatment. The treatment method includes a stent type, a placement position, and a dilation diameter. The grouping result is a result of classification of the found first and second similar cases RS1 and RS2. Here, the similar cases of the patients A and B are classified into the group GR1, and the similar cases of the patients C and D are classified into the group GR2. The found similar cases have a similar feature to the target image G0 obtained by imaging the patient E, who is the patient to be treated. Thus, all the facilities included in the environment information for the similar cases are the facility A, and all the doctors are the doctor A.

The environment information and patient information with numbers 5 to 8 are for the patient E and are all the same. The treatment methods in the rows with numbers 5 to 8 are the treatment methods with numbers 1 to 4, respectively.

The treatment results with numbers 1 to 4 are described in the search diagnosis logs 33A. The treatment results with numbers 5 to 8 are expected treatment results derived by the treatment result derivation unit 23. Specifically, the treatment result with number 5 is derived by the treatment result derivation unit 23 on the basis of the environment information and the patient information for the patient E and the treatment method with number 1. The treatment result with number 6 is derived by the treatment result derivation unit 23 on the basis of the environment information and the patient information for the patient E and the treatment method with number 2. The treatment result with number 7 is derived by the treatment result derivation unit 23 on the basis of the environment information and the patient information for the patient E and the treatment method with number 3. The treatment result with number 8 is derived by the treatment result derivation unit 23 on the basis of the environment information and the patient information for the patient E and the treatment method with number 4.

When the operator clicks on a row with a desired number on the displayed presentation screen 60 by using the input device 15, the post-treatment image included in the similar case is displayed. In FIG. 9, in response to a click on number 1, the post-treatment image 32 for the similar case with number 1 is displayed in the image display region 62. In FIG. 9, the row with number 1 is shaded with diagonal lines to indicate that number 1 is clicked on.

The doctor can refer to, on the presentation screen 60, patient information, a treatment method, and a treatment result for a case similar to the case of the patient E to be given treatment. Further, the doctor can refer to a treatment result expected if the patient E is treated in accordance with the same treatment method as that for the similar case. Accordingly, the doctor can decide on a treatment method by referring to treatment results based on a large number of post-treatment images. After deciding on a treatment method, the doctor can input the treatment method that has been decided on to the text input region 63. As a result, the treatment method is described in the diagnosis log corresponding to the target image G0 for the patient E to be given treatment.

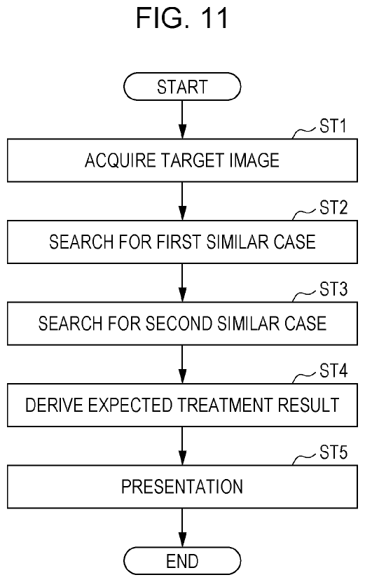

Next, a process performed in this embodiment will be described. FIG. 11 is a flowchart illustrating a process performed in the embodiment. When an instruction is given to start the process, first, the information acquisition unit 21 acquires a target image G0 for a patient to be treated from the image storage server 3 (step ST1). Then, the search unit 22 searches for a first similar case RS1 having a similar feature to the target image G0 and including the post-treatment image 32 from among the plurality of reference cases stored in the image storage server 3 (step ST2). The search unit 22 further searches for a second similar case RS2 having a similar feature to the post-treatment image included in the first similar case RS1 (step ST3).

Then, the treatment result derivation unit 23 derives an expected treatment result expected if the treatment target patient is treated in accordance with the treatment method described in the search diagnosis log 33A (step ST4). Then, the presentation unit 24 presents the contents of the description of the search diagnosis log 33A and the expected treatment result to the operator by displaying the contents of the description and the expected treatment result on the display 14 (step ST5). Then, the process ends.

In this embodiment, as described above, a second similar case RS2 having a similar feature to a post-treatment image included in a first similar case RS1 is searched for, and a treatment method and a treatment result described in a search diagnosis log 33A, which is a diagnosis log 33 included in each of the first and second similar cases RS1 and RS2, are presented. Accordingly, information that fully reflects the effects of various treatment methods on the treatment results can be provided to the operator.

In this embodiment, furthermore, an expected treatment result expected if the treatment target patient is treated in accordance with the treatment method described in the search diagnosis log 33A is derived and presented. Accordingly, the doctor can select a treatment method predicted to provide a satisfactory treatment result.

In some cases, a treatment target patient may be given a plurality of treatments. For example, if the coronary arteries are stenosed at a plurality of locations, stents are placed at the plurality of locations per surgery. In such surgery, when a stent is placed at each location with stenosis, the patient is imaged to check the condition. In this case, it is preferable to repeat a search for the first similar case RS1, a search for the second similar case RS2, and presentation of a treatment result by using an image acquired after the placement of each stent as a new target image G0. This will be described hereinafter as another embodiment.

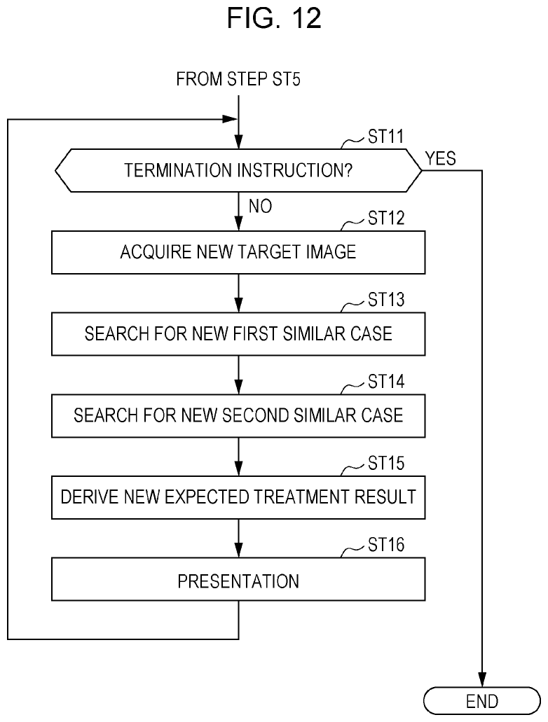

FIG. 12 is a flowchart illustrating a process performed in another embodiment. While the process according to another embodiment is performed during surgery on a patient, the processing up to the presentation of the first treatment result is preferably performed before the surgery. In another embodiment, the processing up to the presentation of the first treatment result is the same as the processing from step ST1 to step ST5 illustrated in FIG. 11. Thus, only the processing after step ST5 in FIG. 11 will be described.

When a treatment result is presented in step ST5 illustrated in FIG. 11, the operator refers to the presented treatment result, decides on a treatment plan for the patient, and gives treatment to the patient. In another embodiment, surgery is performed to place a stent at the first stenosis site in the coronary arteries of the patient. After the surgery for placing a stent at the first stenosis site is performed, the patient is imaged to acquire an image. The acquired image is stored in the image storage server 3 as a new target image G0.

Then, it is determined whether an instruction to terminate the process has been given (step ST11). If step ST11 is negative, the information acquisition unit 21 acquires the new target image G0 from the image storage server 3 (step ST12). Then, the search unit 22 searches for a new first similar case RS1 having a similar feature to the new target image G0 and including the post-treatment image 32 from among the plurality of reference cases stored in the image storage server 3 (step ST13). The search unit 22 further searches for a new second similar case RS2 having a similar feature to the post-treatment image included in the new first similar case RS1 (step ST14). Then, the treatment result derivation unit 23 derives an expected treatment result expected if the treatment target patient is treated in accordance with the treatment method described in a new search diagnosis log 33A (step ST15). Then, the presentation unit 24 presents the contents of the description of the search diagnosis log 33A and the expected treatment result to the operator by displaying the contents of the description and the expected treatment result on the display 14 (step ST16). Then, the process returns to the processing of step ST11. If step ST11 is affirmed, the process ends.

Accordingly, in a case where a plurality of portions of treatment are present, each time treatment is completed for one of the portions of treatment, information that fully reflects the effect of the treatment method on the treatment result can be provided to the operator for a similar case similar to the target image G0 after the treatment is completed.

In the embodiment described above, the description of a diagnosis log for the target image G0 is used as information on a patient to be treated. However, the present disclosure is not limited thereto. Patient information including diagnosis results of the patient, which are input by a doctor interpreting the target image G0, which is displayed on the display 14, may be used.

In the embodiment described above, a CT image is used as the target image G0. However, the present disclosure is not limited thereto. In place of the CT image, a three-dimensional image such as an MM image or a PET image may be used. Alternatively, a radiographic image acquired by simple radiographic imaging of the patient may be used as the target image G0.

In the embodiment described above, furthermore, the target image G0 obtained by imaging a patient with stenosis in a coronary artery is used to perform the process. However, the symptom of the patient is not limited to stenosis in a coronary artery. A target image G0 acquired by imaging a patient with any symptom may be used to decide on a treatment method in accordance with the process according to this embodiment. Further, the treatment method is directed to, but is not limited to, surgery to place a stent. The treatment method may be directed to any treatment method for treating a patient, such as surgery or medication in accordance with the symptom of the patient.

In the embodiment described above, the hardware structures of processing units that execute various processes, such as the information acquisition unit 21, the search unit 22, the treatment result derivation unit 23, and the presentation unit 24, may be implemented using various processors described below. As described above, the various processors described above include a CPU that is a general-purpose processor configured to execute software (program) to function as various processing units, and further include a Programmable Logic Device (PLD) that is a processor whose circuit configuration can be changed after manufacturing, such as an FPGA (Field Programmable Gate Array), a dedicated electric circuit that is a processor having a circuit configuration designed specifically for executing specific processing, such as an ASIC (Application Specific Integrated Circuit), and so on.

A single processing unit may be configured as one of the various processors or as a combination of two or more processors of the same type or different types (for example, a combination of a plurality of FPGAs or a combination of a CPU and an FPGA). Alternatively, a plurality of processing units may be configured as a single processor.

Examples of configuring a plurality of processing units as a single processor include, first, a form in which, as typified by a computer such as a client or a server, the single processor is configured as a combination of one or more CPUs and software and the processor functions as the plurality of processing units. The examples include, second, a form in which, as typified by a system on chip (SoC) or the like, a processor is used in which the functions of the entire system including the plurality of processing units are implemented as one IC (Integrated Circuit) chip. As described above, the various processing units are configured by using one or more of the various processors described above as a hardware structure.

More specifically, the hardware structure of these various processors may be an electric circuit (Circuitry) in which circuit elements such as semiconductor elements are combined.

What is claimed is:

1. A treatment support apparatus comprising at least one processor configured to:
search for a first similar case from among a plurality of reference cases, each of the plurality of reference cases including at least one diagnosed image and a diagnosis log, the diagnosis log describing a treatment method performed on a diagnosed patient for whom the diagnosed image is acquired and a treatment result obtained by the treatment method, the first similar case having a similar feature to a target image obtained by imaging a treatment target patient who is to be treated, the first similar case including, as the diagnosed image, a post-treatment image obtained through imaging after treatment;
search for a second similar case from among the plurality of reference cases, the second similar case having a similar feature to the post-treatment image included in the first similar case; and
present the treatment method and the treatment result described in a search diagnosis log that is the diagnosis log included in each of the first similar case and the second similar case.

2. The treatment support apparatus according to claim 1, wherein the diagnosis log describes doctor-responsible-for-diagnosis information and patient information, the doctor-responsible-for-diagnosis information identifying a doctor who has given treatment to the diagnosed patient, the patient information including a diagnosis result of the diagnosed patient before the treatment, and
the processor is configured to further present the doctor-responsible-for-diagnosis information and the patient information.

3. The treatment support apparatus according to claim 2, wherein the processor is configured to derive an expected treatment result on the basis of treating-doctor information identifying a doctor in charge of treatment for the treatment target patient, patient information including a diagnosis result of the treatment target patient, and the treatment method described in the search diagnosis log, the expected treatment result being a treatment result expected if the doctor in charge of treatment treats the treatment target patient in accordance with the treatment method described in the search diagnosis log.

4. The treatment support apparatus according to claim 3, wherein the processor is configured to derive the expected treatment result by using a trained model, the trained model being constructed by machine learning using training data in which treating-doctor information, patient information, and treatment methods are training information and treatment results are ground-truth data.

5. The treatment support apparatus according to claim 1, wherein in a case where a plurality of portions of treatment are present in the treatment target patient and a new target image is acquired by imaging the treatment target patient each time treatment is performed on one of the portions of treatment,
the processor is configured to repeat a new search for the first similar case, a new search for the second similar case, and presentation of the treatment result each time treatment is performed on one of the portions of treatment.

6. The treatment support apparatus according to claim 1, wherein
the target image is acquired by imaging a treatment target patient having stenosis in a coronary artery,
the treatment method includes a type of a stent, a placement position of the stent, and a dilation diameter of the coronary artery, and
the treatment result is a diameter of the coronary artery after placement of the stent.

7. A treatment support method comprising:
searching for a first similar case from among a plurality of reference cases, each of the plurality of reference cases including at least one diagnosed image and a diagnosis log, the diagnosis log describing a treatment method performed on a diagnosed patient for whom the diagnosed image is acquired and a treatment result obtained by the treatment method, the first similar case having a similar feature to a target image obtained by imaging a treatment target patient who is to be treated, the first similar case including, as the diagnosed image, a post-treatment image obtained through imaging after treatment;
searching for a second similar case from among the plurality of reference cases, the second similar case having a similar feature to the post-treatment image included in the first similar case; and
presenting the treatment method and the treatment result described in a search diagnosis log that is the diagnosis log included in each of the first similar case and the second similar case.

8. A non-transitory computer-readable storage medium that stores a treatment support program for causing a computer to execute the steps of:
searching for a first similar case from among a plurality of reference cases, each of the plurality of reference cases including at least one diagnosed image and a diagnosis log, the diagnosis log describing a treatment method performed on a diagnosed patient for whom the diagnosed image is acquired and a treatment result obtained by the treatment method, the first similar case having a similar feature to a target image obtained by imaging a treatment target patient who is to be treated, the first similar case including, as the diagnosed image, a post-treatment image obtained through imaging after treatment;
searching for a second similar case from among the plurality of reference cases, the second similar case having a similar feature to the post-treatment image included in the first similar case; and
presenting the treatment method and the treatment result described in a search diagnosis log that is the diagnosis log included in each of the first similar case and the second similar case.
